SEQUENCE LISTING (22 sequences)

SEQ ID NO: 1 - DNA, 23 bp, Artificial Sequence
  Description of Artificial Sequence Primer
agacttcgcatggctgtaaccag

SEQ ID NO: 2 - DNA, 22 bp, Artificial Sequence
  Description of Artificial Sequence Primer
ggggaccgtaagagacacacac

SEQ ID NO: 3 - DNA, 24 bp, Artificial Sequence
  Description of Artificial Sequence Primer
gggcgctggattttcaaccttcta

SEQ ID NO: 4 - DNA, 23 bp, Artificial Sequence
  Description of Artificial Sequence Primer
gttatccaactggggaagcagga

SEQ ID NO: 5 - DNA, 25 bp, Artificial Sequence
  Description of Artificial Sequence Primer
gactgctgggaggagacattcagaa

SEQ ID NO: 6 - DNA, 25 bp, Artificial Sequence
  Description of Artificial Sequence Primer
gactgctgggaggagacattcagaa

SEQ ID NO: 7 - DNA, 23 bp, Artificial Sequence
  Description of Artificial Sequence Primer
taatgggagccacgctaataccc

SEQ ID NO: 8 - DNA, 23 bp, Artificial Sequence
  Description of Artificial Sequence Primer
agctccagggaggcaggaactat

SEQ ID NO: 9 - DNA, 24 bp, Artificial Sequence
  Description of Artificial Sequence Primer
gttcaggagcgttttgttttgcag

SEQ ID NO: 10 - DNA, 24 bp, Artificial Sequence
  Description of Artificial Sequence Primer
acccactttgggaaaagctgttgt

SEQ ID NO: 11 - DNA, 25 bp, Artificial Sequence
  Description of Artificial Sequence Primer
tgaggtttgggactcttggtttttc

SEQ ID NO: 12 - DNA, 22 bp, Artificial Sequence
  Description of Artificial Sequence Primer
cttcctccctcctgtgctctga

SEQ ID NO: 13 - DNA, 23 bp, Artificial Sequence
  Description of Artificial Sequence Primer
caagccttgtgagaagcgaacac

SEQ ID NO: 14 - DNA, 24 bp, Artificial Sequence
  Description of Artificial Sequence Primer
tggaggctcctaggtctcaatgaa

SEQ ID NO: 15 - DNA, 23 bp, Artificial Sequence
  Description of Artificial Sequence Primer
caagccttgtgagaagcgaacac

SEQ ID NO: 16 - DNA, 23 bp, Artificial Sequence
  Description of Artificial Sequence Primer
ttaaagaacatgggcacggtgag

SEQ ID NO: 17 - DNA, 22 bp, Artificial Sequence
  Description of Artificial Sequence Primer
ctgacttccagaggtcctggtc

SEQ ID NO: 18 - DNA, 23 bp, Artificial Sequence
  Description of Artificial Sequence Primer
ttaaagaacatgggcacggtgag

SEQ ID NO: 19 - DNA, 24 bp, Artificial Sequence
  Description of Artificial Sequence Primer
gtgaactcttttcgcaagctggtt

SEQ ID NO: 20 - DNA, 23 bp, Artificial Sequence
  Description of Artificial Sequence Primer
aagctagtgcctgtggggaaatc

SEQ ID NO: 21 - DNA, 25 bp, Artificial Sequence
  Description of Artificial Sequence Primer
gtgaaaagtgtccttgttgttcatg

SEQ ID NO: 22 - DNA, 24 bp, Artificial Sequence
  Description of Artificial Sequence Primer
caactctatcacaccagcttgagc

(19) United States
(12) Patent Application Publication
(10) Pub. No.: US 2004/0072230 A1
(43) Pub. Date: Apr. 15, 2004

(54) Human SORBS1 genetic variations contribute to insulin resistance, obesity, type 2 diabetes, and hypertension

(76) Inventors: Chao Agnes Hsiung, Taipei (TW); Lee-Ming Chuang, Hsin-Tien City (TW); Chin-Fu Hsiao, Liucha (TW); Tong-Yuan Tai, Taipei (TW)

Correspondence Address: Finnegan, Henderson, Farabow,, Garrett & Dunner, L.L.P., 1300 I Street, N.W., Washington, DC, 20005-3315, US

(21) Appl. No.: 10/639,491

(22) Filed: Aug. 13, 2003

Related U.S. Application Data
(60) Provisional application No. 60/402,911, filed on Aug. 14, 2002.

(51) Int. Cl. C12Q 1/68

(52) U.S. Cl. 435/6

(57) ABSTRACT

The present invention identifies mutations in the human sorbin and SH3-domain-containing-1 (SORBS1) gene, particularly at one or more of positions 220; 249; −7 with respect to exon 5; −25 with respect to exon 6; 682; +64 with respect to exon 9; +61 with respect to exon 10; +69 with respect to exon 11; +33 with respect to exon 16; 1482; 1518; −6 with respect to exon 22; +79 with respect to exon 24; and 2337. A polymorphism at position 682 within exon 7, the T228A allele, correlates with the phenotypes of low blood pressure and low body mass index, and is a protective marker for obesity, diabetes, and hypertension. The invention also relates to methods and materials for detecting single nucleotide polymorphisms in the SORBS1 gene and to the use of SORBS1 polymorphisms in the diagnosis, screening, and treatment of type 2 diabetes, obesity, hypertension, atherosclerosis, and metabolic syndrome.

### CROSS-REFERENCE TO RELATED APPLICATIONS

This application claims priority under 35 U.S.C. § 119(e) of provisional application No. 60/402,911, filed Aug. 14, 2002, the entire disclosure of which is incorporated by reference.

### FIELD OF THE INVENTION

The present invention relates to polymorphisms in the SORBS1 gene. This invention also relates to methods and materials for analyzing allelic variations in the SORBS1 gene, and to the use of SORBS1 polymorphisms in the diagnosis of, predisposition to, and treatment of, disorders of insulin metabolism such as metabolic syndrome, type 2 diabetes, obesity, hypertension, and atherosclerosis.

### BACKGROUND

Single nucleotide polymorphisms, i.e., SNPs, are the cause of most common genetic disorders; as such, they can be utilized to assess an individual's predisposition to disorders, develop genetic medicine, and design personally-tailored therapies (Collins, et al., 1997). All humans have variability at SNP sites, but those with certain polymorphisms are predisposed to have either a greater or lesser risk for a particular disease with a particular genetic component. Many SNPs have no immediate effect on cell function, but can predispose individuals to a disease or influence their response to a drug. For example, SNPs can have a major impact on how individuals respond to insults such as bacteria, viruses, toxins, and chemicals.

In addition to their value in explaining the basis of inheritable variation in susceptibility to disease, there is a growing body of evidence that the fact that individuals react differently to pharmaceutical drugs is due to polymorphisms in the genes that are the targets of the drugs and that these polymorphisms determine individual drug reactions. The science of pharmacogenetics is based upon identification of the basis for inheritable variation in responses among various groups of patients; it is the foundation for rational drug design (Hess and Cooper, 1999).

SNPs occur in both coding and noncoding regions of the genome. SNPs found in both coding and non-coding regions can influence a patient's predisposition to disease and pattern of responding to environmental factors, including pharmaceutical treatment modalities. For example, SNPs in the coding region of the genome can directly alter protein function, and SNPs in the non-coding region of the genome, e.g., those present in the gene's promoter region, can influence gene expression. Therefore SNPs are useful in diagnosing an individual's predisposition to a disease, provide a basis for choosing among available treatment options, and serve as the foundation for the molecular design of new therapeutics.

Disorders of insulin metabolism are involved in the pathogenesis of many common disorders, including type 2 diabetes, obesity, hypertension, and atherosclerosis. The constellation of these metabolic disorders is appreciated as “metabolic syndrome,” a disease characterized by a state of altered glucose tolerance, plasma lipid levels, blood pressure, and body fat distribution (Lopez-Candales, 2001). Metabolic syndrome is a multifaceted clinical entity caused by a combination of genetic, hormonal, and lifestyle factors. It occurs frequently in the general population, and is manifest by truncal obesity, glucose intolerance/diabetes, dyslipidemia, or essential hypertension. Simultaneous presentation of all the components of the syndrome is uncommon, and most experts consider three of the four components sufficient to define the syndrome (Hauner, 2002). Metabolic syndrome precedes the onset of diabetes in a substantial number of patients.

Diabetes is a chronic disorder of carbohydrate, fat, and protein metabolism. It is a complex disease, with multiple clinical variants. Both hereditary and environmental factors contribute significantly to the development of the disease. Diabetes is characterized by a decreased ability of the insulin receptor to facilitate glucose uptake into the cell, where it is used as an energy source. The resulting intracellular glucose deficiency increases appetite, resulting in obesity in some patients with type 2 diabetes. In diabetes, the insulin receptor also responds abnormally to insulin in its role in regulating lipid metabolism; as a result, plasma lipid levels increase. The elevated plasma lipids present in chronic diabetes generally include elevated plasma cholesterol and triglyceride concentrations and reduced high density lipoprotein cholesterol (HDLc), and thus can play a role in the genesis of atherosclerosis, which is present in diabetic patients to a greater extent than in the general population.

Diabetes poses a significant public health problem. There are over 16 million diabetics in the United States, and over 1 million in Taiwan. Both type 1 and type 2 diabetes increase the risk of cardiovascular disease, retinopathy, neuropathy, and nephropathy, which can lead to heart attacks, strokes, blindness, renal insufficiency, and amputation.

Insulin resistance and concomitant hyperinsulinemia commonly contribute to the pathogenesis of both type 2 diabetes and essential hypertension. Attempts to identify genes responsible for hypertension in large population groups were unsuccessful (Province et al., 2000; Ranade et al., 2000) until recently, when single nucleotide polymorphisms (SNPs) in the human urea transporter-2 and the β₂-adrenergic receptor were demonstrated to be associated with variations in blood pressure (Ranade 2001a; Ranade et al., 2001b).

Hypertension can be linked to obesity, diabetes, and the metabolic syndrome through a common central neuroendocrine origin. In obesity and diabetes, peripheral endocrine perturbations act as triggers for an enhanced engagement of the neuroendocrine hypothalamic-pituitary-adrenal axis. The limbic-hypothalamic pathway is known to affect blood pressure. These two neuroendocrine pathways are tightly coupled functionally, and their signals to the body's periphery are often combined. For example, both pathways mediate the human's primitive survival response to stress. Over the long-term, events that occur along both the hypothalamic-pituitary-adrenal axis and the limbic-hypothalamic pathway appear to be decisive in the development of primary hypertension and the metabolic syndrome. Susceptibility to these disorders is a combination of environmental and genetic factors (Bjorntorp et al., 2000).

Obesity has multiple contributing factors, both genetic and environmental. Genes that affect the risk for human obesity include SORBS1, PPARA, PPARG2, VCP1, VCP2, VCP3, BAR-Z, APMI, and leptin (Nieters et al., 2002). Environmental factors include diet and physical activity. Obesity is characterized by the occurrence of adipogenesis, which is the deposition of fat, involving conversion of carbohydrate or protein to fat.

Insulin binding to the extracellular surface of the insulin receptor changes the conformation of the receptor. The conformational change in the receptor, a transmembrane protein, causes the phosphorylation of catalytic domains within the receptor, which in turn results in the phosphorylation of a substrate protein, namely, insulin receptor substrate-1. The phosphorylation of the receptor substrate turns it into a high-affinity binding site for the docking and activation of intracellular signaling proteins. The resulting cascade of intracellular signals mediates the regulation of glucose and lipid metabolism by the insulin receptor. One of the regulatory proteins in this signaling cascade is human SORBS1, the subject of the present invention.

### SUMMARY OF THE INVENTION

The present invention addresses the need in the art for a better understanding of the genetic factors that contribute to disorders of insulin metabolism, such as metabolic syndrome, type 2 diabetes, obesity, hypertension, and atherosclerosis. The invention addresses this need by identifying single nucleotide polymorphisms (SNPs) in the human SORBS1 gene that correlate with a predisposition, or lack thereof, for these disorders.

In general, the invention provides for the identification of SNPs in the human SORBS1 gene, and their use in the diagnosis of a predisposition towards, and the treatment of, disorders of insulin metabolism. Six SNPs were found in the coding region of the gene, and eight were found in the intronic regions of the genome, within 79 base pairs of the coding region. These SNPs are located at positions 220 (within exon 4); 249 (within exon 4); −7 with respect to exon 5; −25 with respect to exon 6; 682 (corresponding to the T228A polymorphism within exon 7); +64 with respect to exon 9; +61 with respect to exon 10; +69 with respect to exon 11; +33 with respect to exon 16; 1482 (within exon 18); 1518 (within exon 18); −6 with respect to exon 22; +79 with respect to exon 24; and 2337 (within exon 26), based on the GenBank accession numbers AF136380, AF136381, and AF356525-AF356527 for the alternatively spliced SORBS1 gene. Among these, two SNPs affected amino acid coding, R74W and T228A. Further studies associated the T228A polymorphism with type 2 diabetes, obesity hypertension, atherosclerosis, and metabolic syndrome. Variations in the human SORBS1 gene play a role in controlling variations of insulin resistance and the risks for associated diseases.

### DETAILED DESCRIPTION OF THE INVENTION

Definitions

As used herein, “single nucleotide polymorphism,” or “SNP” is a DNA sequence variation that occurs when a nucleotide, e.g., adenine (A), thymine (T), cytosine (C), or guanine (G), in the genome sequence is altered to another nucleotide. SNPs are occasional variations in gene sequence; the vast majority of the DNA sequence is identical among all humans. They represent a genomic hot spot responsible for the genetic variability among humans.

As used herein, “gene” means any amount of nucleic acid material that is sufficient to encode a protein having the function desired. Thus, it includes, but is not limited to, genomic DNA, cDNA, RNA, and nucleic acid that is otherwise genetically engineered to achieve a desired level of expression under desired conditions. Accordingly, it includes fusion genes (encoding fusion proteins), intact genomic genes, and DNA sequences fused to heterologous promoters, operators, enhancers, and/or other transcription regulating sequences. Methods and nucleic acid constructs for preparing genes for recombinant expression are well known and widely used by those of skill in the art, and thus need not be detailed here.

As used herein, an “exon” is a segment of a eucaryotic gene that encodes a sequence of nucleotides in mRNA. An exon can encode amino acids in a protein. Exons are generally adjacent to introns.

As used herein, an “intron” is a non-coding region of a eucaryotic gene that may be transcribed into an RNA molecule, but is not translated into amino acids. It may be excised by RNA splicing when mRNA is produced.

As used herein, a “patient” is any living animal, including, but not limited to, a human who has, or is suspected of having or being susceptible to, a disease or disorder, or who otherwise would be a subject of investigation relevant to a disease or disorder. Accordingly, a patient can be an animal that has been bred or engineered as a model for metabolic syndrome, type 2 diabetes, obesity, hypertension, atherosclerosis, or any other disease or disorder. Likewise it can be a human suffering from, or at risk of developing, a disease or disorder associated with insulin metabolism, or any other disease or disorder. Similarly, a patient can be an animal (such as an experimental animal, a pet animal, a farm animal, a dairy animal, a ranch animal, or an animal cultivated for food or other commercial use), including a human, serving as a healthy control for investigations into diseases and/or disorders, e.g., those associated with insulin metabolism.

By “reagent,” it is meant any element, molecule, or compound that is present in the assay system and participates, either directly or indirectly, in the biochemical processes occurring during the performance of the method. Reagents include, but are not limited to, nucleic acids, cells, media, chemicals, compounds used to introduce nucleic acids into cells, and compounds used to generate detectable signals.

By “materials,” it is meant items that are used to contain and/or perform the methods of the invention, but that do not participate in any of the biochemical reactions taking place in the method. Materials include, but are not limited to, test tubes, pipettes, gels, and ultraviolet transilluminators.

The Human SORBS1 Gene

The human SORBS1 gene is a single-copy gene that is ubiquitously expressed in all human tissues, but is most abundant in heart and skeletal muscle (Lin et al., 2001a). This gene maps to human chromosome 10q23.3-q24.1 (Lin et al., 2001a), a region of chromosome 10 identified to contain a linkage marker for type 2 diabetes in individuals of European descent (Wiltshire et al., 2001). It was identified by cloning and sequencing several cDNAs with various alternatively spliced exons (Lin et al., 2001a). The GenBank accession numbers of these cDNAs are AF136380, AF136381, and AF356525-AF356527. The intron/exon boundaries and the sizes of the introns of the SORBS1 gene were determined by alignment with the genomic sequences AL158165 and AL160288, obtained from the National Center for Biotechnology Information (NCBI) (Lin, et al., 2001b). Based on the homology and alignment search, the coding region of SORBS1 has been determined to be encoded in 34 exons, separated by 33 introns that span a region of more than 120 kilobases (Lin, et al., 2001b). The sorbin domain is encoded by exons 11, 16, 17, and 18. The SH3 domaains are encoded by exons 26, 27, 28, 29, 32, 33, and 34 (Lin et al., 2001a).

The human SORBS1 gene encodes an adaptor protein in the insulin-signaling pathway (Yang et al., 2003). The SORBS1 (sorbin and SH3 domain containing 1) protein is a human homologue of murine CAP/ponsin. Sequence comparison reveals an 88% homology in amino acid sequence between the mouse and human proteins (Lin et al., 2001b).

Like its murine analogue CAP/ponsin, human SORBS1 is recognized as a functional component of the insulin-stimulated signaling pathway. In the absence of insulin stimulation, human SORBS1 associates with the insulin receptor in the human hepatoma cell line Hep3B. After insulin stimulation, SORBS1 dissociates from the insulin receptor and binds to c-Abl, an intracellular signaling protein and effector of insulin action, via its third SH3 domain (Lin et al., 2001a). Therefore, the SORBS1 gene product regulates insulin action, and variants in the gene can be expected to cause variations in insulin metabolism.

The human SORBS1 gene encodes a member of the vinexin family of adaptor proteins, which link appropriate proteins to other proteins or to membranes. Adaptor proteins are instrumental in constructing large signaling complexes, and can therefore specify the subcellular localization of specific molecules (Kioka et al., 2002). The vinexin family of adaptor proteins is involved in signal transduction and cytoskeletal organization. Its members contain a sorbin homology domain and three src homology 3 (SH3) domains in their C-terminal region. The sorbin homology domain was named for its a high degree of homology to sorbin, an active peptide that was originally purified from porcine intestine and found to stimulate absorption of water and electrolytes in the guinea pig gall bladder (Charpin et al., 1992). The SH3 domain is a highly conserved sequence found on intracellular signaling proteins, including adaptor proteins. These domains couple phosphorylated proteins to other proteins within signaling complexes (Mayer, 2001; Alberts et al., 1994).

SORBS1 is involved in insulin signaling through its association with the insulin receptor. In the unstimulated basal state, SORBS1 is associated with the insulin receptor, and co-immunoprecipitates with the receptor β-subunit; insulin stimulation dissociates SORBS1 from the insulin receptor complex (Lin et al., 2001a). After insulin stimulation, the amount of SORBS1 binding to the cAbl oncoproteins is increased; this binding is mediated by the third SH3 domain of SORBS1 (Lin et al., 2001a).

Murine CAP/Ponsin

Murine CAP/ponsin, the protein product of the SH3P12 gene, and a relative of the human SORBS1 gene, was first identified as a member of the vinexin family of adaptor proteins by a functional screen for binding to an SH3 ligand, and was first named SH3P12 (Sparks et al., 1996). It was subsequently found to be a ligand for the signaling protein c-Cbl and the adaptor protein afadin (Mandai et al., 1999; Ribon et al., 1998a). It is recognized as an adaptor protein with roles in signal transduction and cytoskeletal rearrangement (Zhang et al., 2003).

CAP/ponsin has recently been recognized as an important regulatory protein in the insulin-stimulated signaling pathway. Insulin exerts its physiologic effect through the insulin receptor, which acts, in part, by stimulating the translocation of glut4, an insulin-responsive glucose transporter, from intracellular storage vesicles to the plasma membrane, leading to increased glucose uptake into adipose tissue and muscle (Kioka et al, 2002). CAP/ponsin interacts with the insulin receptor to regulate insulin-stimulated glucose uptake (Baumann et al., 2000).

Insulin initiates its actions by binding to the insulin receptor, resulting in the phosphorylation of intracellular substrates. An example of an intracellular insulin receptor substrate is c-Cbl, a signaling molecule and CAP/ponsin ligand. Insulin-stimulated phosphorylation of the insulin receptor induces the c-Cbl/CAP/ponsin complex to dissociate from the receptor and relocate to intracellular caveolae. In the caveolae, another protein, flotillin, joins the complex (Bickel et al., 1997), which then relocates further to a lipid raft subdomain of the plasma membrane. The signaling molecules on the lipid raft exert their effects on downstream targets of the insulin receptor, and the physiologic effect is glucose uptake into the cell (Ribon et al., 1998a; Baumann et al., 2000).

CAP/ponsin enhances insulin-induced protein phosphorylation, and plays a role in assembling lipid raft domains, where groups of signaling molecules accumulate (Simons and Toomre, 2000). The sorbin homology domain and the SH3 domains play a role in this signaling. Both the sorbin homology domain and the SH3 domains of CAP/ponsin are necessary to permit normal relocation of the signaling ligand c-Cbl to caveolae or lipid rafts, and, in turn, for normal glut4 translocation (Baumann et al., 2000; Kimura et al., 2001).

CAP/ponsin expression in adipose tissues is enhanced by the anti-diabetic thiazolidinedione compound BRL49653 (Ribon et al., 1998b). Thiazolidinedione compounds improve insulin sensitivity in both humans and other animals, and reverse many of the clinical manifestations of type 2 diabetes. They bind to and activate the nuclear peroxisome proliferator activated receptor γ (PPARγ) (Lin et al, 1999), which in turn stimulates the SH3P12 gene to express CAP/ponsin. The human homologue of CAP/ponsin is SORBS1 (sorbin and SH3 domain containing 1).

Regulation of Insulin Metabolism by Differential Gene Expression

The present invention provides, for the first time, a method to identify genes that are differentially expressed during the differentiation of pre-adipocytes to adipocytes, and to study the effect of pharmaceutical agents on the regulation of these genes. Genes that are differentially expressed during this process are involved in adipogenesis. Insulin sensitization takes place during adipogenesis, therefore studying the differential expression of genes during adipogenesis can illuminate the pathogenesis of insulin resistance, including obesity-associated insulin resistance and development of type 2 diabetes. This can provide a method for identifying drugs that affect the regulation of genes involved in insulin resistance.

Murine 3T3-L1 cells can be induced to differentiate from pre-adipocytes to adipocytes in culture. Using mRNA differential display analysis, and confirmed by Northern blot, 56 genes were identified as differentially expressed during differentiation of 3T3-L1 cells to adipocytes. Among these 56 genes, 40 were up-regulated and 16 were down-regulated during adipocyte differentiation. The differentially regulated genes function in a wide array of diverse biological functions, including DNA remodeling, transcription, cytoskeletal function, extracellular matrix function, protein secretion, mitochondrial function, signal transduction, lipid metabolism, and insulin function. They include, e.g., SH3P12/CAP, G protein signaling regulator 2, acid labile subunit, mouse ufo/Ax1, ALS, homeodomain-interacting protein kinase 2, serum deprivation response, ezrin, peg3/pw1, carboxylesterase, ALDR, GM2 activator protein, GPDH, LPL, Sycp3, FAT10, CCR4, PG-M, fibronectin, FAT/CD36 antigen, A10 gene, ASP/complement component 3, proliferin, mouse C10 gene, and adipoQ, the latter of which is inversely correlated with body mass, and is up-regulated by BRL49653. These genes display different patterns of expression. Some are consistently up-regulated through all phases of differentiation, some are up-regulated during specific phases of differentiation, e.g., early or late phases, some are up-regulated in some phases, and down-regulated in other phases, and some are consistently down-regulated through all phases of differentiation.

Expression of the SH3P12 gene, which encodes CAP/ponsin, the murine homologue of SORBS1, was consistently increased during adipocyte differentiation. The increase was apparent on day 1 of induction, and the level of expression was approximately twice as high on day 10 as on day 1. The increased level of SH3P12 gene expression correlated with, and was immediately preceded by, an increase in expression of the transcriptional factor PPARγ. SH3P12/CAP is up-regulated during 3T3-L1 adipocyte differentiation; this up-regulatin was further enhanced by BRL49653 stimulation.

The anti-diabetic agent BRL49653 (rosiglitazone) stimulates adipocyte differentiation. It augments insulin action by enhancing insulin stimulated glucose uptake in adipocytes, and decreases plasma glucose levels in some type 2 diabetics. BRL49653 also regulates the differential expression of the murine SH3P12 gene during differentiation of 3T3-L1 pre-adipocytes to adipocytes. BRL49653 enhances the induction of the SH3P12 gene. In the absence of BRL49653, the level of SH3P12/CAP/ponsin mRNA on day 6 post-induction was 8.25 fold higher than preinduction levels. When BRL49653 was added to well-differentiated adipocytes on day 6, the level of SH3P12/CAP mRNA was further increased to 17.6 fold higher than pre-induction levels. Thus, CAP/ponsin is induced by BRL49653, and is a mediator of the pharmacological actions of BRL49653. In the murine adipocyte differentiation model system, an increase in the signal protein CAP/ponsin reflects a physiologic augmentation of insulin function.

SORBS1 SNPs

This invention provides for the first time that SNPs in the human SORBS1 gene can diagnose an individual's predisposition for a disorder of insulin metabolism, provide a basis for choosing among existing treatment options, and serve as a foundation for the molecular design of new therapeutics to treat disorders of insulin metabolism.

Individuals who carry particular allelic variants of the SORBS1 gene can exhibit differences in their ability to regulate insulin metabolism under different physiological conditions and in their ability to react to environmental conditions. In addition, differences in insulin metabolism arising as a result of allelic variation can have a direct effect on the response of an individual to drug therapy. SORBS1 polymorphisms can therefore have the greatest effect on the efficacy of drugs designed to modulate the activity of the SORBS1 gene product or other components of the insulin receptor signaling pathway. Additionally, polymorphisms can also affect a patient's response to agents acting on biochemical pathways, other than the insulin receptor pathway, that interact with SORBS1. The diagnostic methods of the invention can therefore be useful both to predict the clinical response to such agents and to determine optimal therapeutic dosing information.

The SORBS1 gene and its gene products are also useful in identifying genes or gene products that interact with SORBS1 gene products. Various methods for identifying such genes or gene products are known in the art. Well known techniques include, but are not limited to, yeast two hybrid systems, immunoprecipitation under non-denaturing conditions, and receptor binding assays.

The SNPs identified in the human SORBS1 gene provide useful tools for developing new treatments for disorders of insulin metabolism. Some mutations in a gene that regulates intracellular insulin signaling can be predicted to result in variants with disrupted gene regulation or disrupted protein. Accordingly, the SORBS1 gene, its gene products, agents that mimic the SORBS1 gene product or its gene products, or agents that inhibit or disrupt the function of the SORBS1 gene or its gene products, can be useful in treating disorders of insulin metabolism. Such agents can be identified by routine screening assays that examine levels of the gene or gene product. Agents that alter levels and/or expression of the gene or gene product are expected to be useful for treating disorders of insulin metabolism.

In one aspect, the invention provides a method for identifying a SNP in the SORBS1 gene of a patient by determining the sequence of the nucleic acid of the patient at one or more of positions 220 (within exon 4); 249 (within exon 4); −7 with respect to exon 5; −25 with respect to exon 6; 682 (corresponding to the T228A polymorphism within exon 7); +64 with respect to exon 9; +61 with respect to exon 10; +69 with respect to exon 11; +33 with respect to exon 16; 1482 (within exon 18); 1518 (within exon 18); −6 with respect to exon 22; +79 with respect to exon 24; and 2337 (within exon 26); as defined by the GenBank accession numbers AF136380, AF136381, and AF356525-AF356527 for the alternatively spliced SORBS1 gene, and determining the status of the human by reference to the polymorphism in the SORBS1 gene.

In another aspect, the methods for detecting a polymorphism, diagnosing a predisposition to an insulin disorder, and providing treatments described herein, are ones in which the SNP at position 220 is the presence of G and/or T.

In another aspect, the methods for detecting a polymorphism, diagnosing a predisposition to an insulin disorder, and providing treatments described herein, are ones in which the SNP at position 249 is the presence of A and/or G.

In another aspect, the methods for detecting a polymorphism, diagnosing a predisposition to an insulin disorder, and providing treatments described herein, are ones in which the SNP at position −7 with respect to exon 5 is the presence of C and/or T.

In another aspect, the methods for detecting a polymorphism, diagnosing a predisposition to an insulin disorder, and providing treatments described herein, are ones in which the SNP at position −25 with respect to exon 6, is the presence of A and/or G.

In another aspect, the methods for detecting a polymorphism, diagnosing a predisposition to an insulin disorder, and providing treatments described herein, are ones in which the SNP at position 682 is the presence of A and/or G.

In another aspect, the methods for detecting a polymorphism, diagnosing a predisposition to an insulin disorder, and providing treatments described herein, are ones in which the SNP at position +64 with respect to exon 9 is the presence of C and/or T.

In another aspect, the methods for detecting a polymorphism, diagnosing a predisposition to an insulin disorder, and providing treatments described herein, are ones in which the SNP at position +61 with respect to exon 10 is the presence of C and/or T.

In another aspect, the methods for detecting a polymorphism, diagnosing a predisposition to an insulin disorder, and providing treatments described herein, are ones in which the SNP at position +69 with respect to exon 11 is the presence of T and/or C.

In another aspect, the methods for detecting a polymorphism, diagnosing a predisposition to an insulin disorder, and providing treatments described herein, are ones in which the SNP at position +33 with respect to exon 16 is the presence of C and/or T.

In another aspect, the methods for detecting a polymorphism, diagnosing a predisposition to an insulin disorder, and providing treatments described herein, are ones in which the SNP at position 1482 is the presence of T and/or C.

In another aspect, the methods for detecting a polymorphism, diagnosing a predisposition to an insulin disorder, and providing treatments described herein, are ones in which the SNP at position 1518 is the presence of C and/or T.

In another aspect, the methods for detecting a polymorphism, diagnosing a predisposition to an insulin disorder, and providing treatments described herein, are ones in which the SNP at position −6 with respect to exon 22 is the presence of G and/or T.

In another aspect, the methods for detecting a polymorphism, diagnosing a predisposition to an insulin disorder, and providing treatments described herein, are ones in which the SNP at position +79 with respect to exon 24 is the presence of C and/or T.

In another aspect, the methods for detecting a polymorphism, diagnosing a predisposition to an insulin disorder, and providing treatments described herein, are ones in which the SNP at position 2337 is the presence of G and/or A.

Predisposition, Diagnosis, and Treatment of Insulin Disorders

Two SNPs in the SORBS1 gene affect amino acid coding, namely the R74W polymorphism and the T228A polymorphism. The T228A polymorphism is associated with a decreased predisposition to type 2 diabetes, obesity, hypertension, atherosclerosis, and metabolic syndrome. The R74W polymorphism did not correlate with diabetes or obesity (Lin et al., 2001b).

The molecular variant R74W in exon 4 results from a cytosine to thymidine change, which substitutes arginine (CGG) with tryptophan (TGG). The genotypic frequencies associated with the R74W polymorphism were determined in control, diabetic, and obese patients using a PCR-based denaturing high performance liquid chromatography (DHPLC) assay employing the WAVE® Nucleic Acid Fragment Analysis System (Transgenomic, Inc., San Jose, Calif.). The frequencies of R74W heterozygotes were not different among 480 patients included in three groups of subjects (8.2% for control patients; 6.0% for diabetic patients; and 3.5% for obese patients.) No homozygous variant was detected.

The molecular variant T228A in exon 7 results from an adenine to guanine change, which substitutes threonine (ACG) with alanine (GCG). The allelic frequency for the variant Ala allele was 0.075 in 40 chromosomes (Lin et al., 2001b). The genotypic frequency and allele frequency of the T228A polymorphism was studied in a cohort of 1796 patients from 740 families of Chinese and Japanese origins, recruited in the San Francisco Bay Area, Hawaii, and Taiwan. This cohort included hypertensive probands, their parents, and their siblings. The genotype and allele frequencies of T228A polymorphism of the SORBS1 gene were significantly different in Japanese compared to Chinese patients (p<0.0001). More Japanese study participants, including hypertensive and non-hypertensive, had the TT genotype, and more Chinese study participants had the AA genotype. The genotype and allele frequencies of the T228A polymorphism in the hypertensive probands also differed significantly between Japanese and Chinese patients (p<0.0001) (Lin et al., 2001b).

Thus, the higher frequency of the A allele in the Chinese, as compared to the Japanese population, can have important prognostic and therapeutic implications for the development and use of antihypertensive drugs in individuals of Chinese and Japanese origin. Accordingly, in general, differences in SORBS1 gene allele frequencies can have important prognostic and therapeutic implications for the diagnosis of disorders of insulin metabolism, and the development and use of pharmaceuticals to treat these disorders.

The T228A polymorphism correlates with the phenotype of low body mass. In a group of 315 non-diabetic subjects, individuals with the TA or AA genotype had a lower body mass index (BMI), a measure of obesity, as compared to individuals with the TT genotype. Individuals with the TA or AA genotype also had a lower fasting plasma insulin concentration than individuals with the TT genotype, and a lower insulin resistance. No differences were observed in fasting plasma glucose concentration, or two hour plasma glucose concentration after oral glucose challenge. Covariate analysis determined that the primary impact of the T228A polymorphism is on body mass index, which is one determinant of insulin resistance (Lin et al., 2001b).

The A allele of the T228A polymorphism is a protective marker for both obesity and diabetes. The frequency of heterozygous or homozygous variants (T/A or A/A genotype) is significantly lower in obese and diabetic patients as compared to normal controls. Non-obese patients have almost twice the frequency of the variant A allele as the obese group (Lin et al., 2001b).

A study of 602 families indicated that the T228A polymorphism correlated with blood pressure, and that it exerted its effect through a recessive mode. The risk for hypertension was significantly lower in siblings with the AA genotype (adjusted OR=0.18; p=0.0136). Patients homozygous for the AA genotype had a diastolic blood pressure 7 mm Hg lower and mean arterial pressure 10 mm Hg lower than their siblings with discordant genotypes (TT or TA) after adjustment for age, gender, and BMI. A sibling-based transmission-disequilibrium test (S-TDT) in a sample subset of 220 pedigrees with marker data information from unaffected siblings, satisfying the minimal structure, further confirmed the contribution of the T228A polymorphism of the SORBS1 gene to hypertension. There was a significant linkage between this locus and hypertension (Z score=3.178, p=0.000742).

The methods for detecting a polymorphism, diagnosing a predisposition to an insulin disorder, and providing treatments described herein, are preferably ones in which the sequence is determined by the polymerase chain reaction and/or restriction fragment length polymorphisms.

In a further aspect, the invention provides a method for diagnosing the predisposition to a disorder of insulin metabolism by identifying SNPs in the human SORBS1 gene that predict a risk for a present or future disorder of insulin metabolism that differs (may be higher or lower) from the risk level in a defined population, e.g., the general population. The method encompasses obtaining nucleic acid from an individual, detecting the presence or absence of a variant nucleotide at one or more of the positions listed above, and determining the status of the individual by reference to polymorphisms in the SORBS1 gene.

Biological samples obtained from an individual can be analyzed for the presence of a variant gene product expressed by the SORBS1 gene to ascertain the individual's susceptibility to disorders of insulin metabolism. Methods for detecting the presence or absence of a known polypeptide sequence are well known in the art. Examples of such methods include, but are not limited to polymerase chain reaction (PCR), ligase chain reaction (LCR), and nucleic acid sequence based amplification (NASBA). Reverse transcriptase PCR (RT-PCR) can also detect the presence of specific mRNA populations in a complex mixture of RNA species. Hybridization to nucleotides arrayed on a solid support can also be used to detect the presence of a selected nucleotide sequence.

The SORBS1 gene product or variants and fragments thereof can be used to raise antibodies against the SORBS1 gene product or variant thereof. Such antibodies can then be used in various assays to detect the presence or absence of the SORBS1 gene product or variant thereof in a sample. Examples of these assays include, but are not limited to, radioimmunoassays, immunohistochemistry assays, competitive-binding assays, Western Blot analyses, ELISA assays, proteomic approaches, two-dimensional gel electrophoresis (2D electrophoresis), and non-gel based approaches such as mass spectrometry or protein interaction profiling. In these methods, the presence or absence of a variant SORBS1 gene product is indicative of the presence of, or the susceptibility to developing a disorder of insulin metabolism.

The invention provides a diagnostic kit comprising an allele-specific oligonucleotide primer pair and appropriate packaging and instructions for use. Such kits can also include appropriate buffer(s) and polymerase(s) such as thermostable polymerases, for example taq polymerase.

The invention also provides a method for predicting which existing therapies are most effective for treating patients with a known genotype with reference to polymorphisms in the SORBS1 gene. The method comprises obtaining nucleic acid from the patient, detecting the presence or absence of a variant nucleotide at one or more of the positions listed above, analyzing interactions between the individual's genotype and the medications used in existing therapies.

The invention further provides a method for designing new therapies effective for treating patients with a known genotype with reference to polymorphisms in the SORBS1 gene. This method selectively targets one or more allelic variants. The method employs screening assays routinely used in pharmaceutical development which examine levels of the SORBS1 gene or gene products. Agents identified as altering levels of and/or expression of the SORBS1 gene or gene product(s) can be useful in treating disorders of insulin metabolism.

The invention provides a method for treating a patient with a drug that affects insulin metabolism by detecting of a SNP in the SORBS1 gene, determining the status of the patient with reference to polymorphisms in the SORBS1 gene, and administering an effective amount of a drug that directly or indirectly affects insulin metabolism.

The invention also provides useful tools for developing new treatments for disorders of insulin metabolism. The SORBS1 gene product, agents which mimic SORBS1 gene products and agents that inhibit disruption in the function of SORBS1 gene products can be useful in treating these disorders. Additionally, agents that alter expression and/or levels of the SORBS1 gene and/or its gene products can also be useful in treating of insulin disorders. Such agents can be identified in routine screening assays which examine levels of the SORBS1 gene or gene product. Agents identified as altering levels and/or expression of the gene or gene product are expected to be useful in the treatment of disorders of insulin metabolism.

In another aspect, the invention provides a pharmaceutical pack comprising a drug that affects insulin metabolism and instructions for administration of the drug to humans diagnostically tested for a SNP in the SORBS1 gene.

In yet another aspect, the invention provides a method for using SNPs in the SORBS1 gene as genetic markers for chromosome region 10q23.3-q24.1 in linkage studies. The SORBS1 gene has been mapped to chromosome 10q23.3-q24.1, a candidate region for inulin sensitivity found in Pima Indians (Lin, 2001b).

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims. Moreover, advantages described in the body of the specification, if not included in the claims, are not per se limitations to the claimed invention.

It is to be understood that both the foregoing general description and the following detailed description are exemplary and explanatory only and are not restrictive of the invention, as claimed. Moreover, it must be understood that the invention is not limited to the particular embodiments described. Further, the terminology used to describe particular embodiments is not intended to be limiting, since the scope of the present invention will be limited only by its claims.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test the invention. Further, all publications mentioned herein are incorporated by reference.

It must be noted that, as used herein and in the appended claims, the singular forms “a,” “or,” and “the” include plural referents unless the context clearly dictates otherwise. Thus, for example, reference to “a subject polypeptide” includes a plurality of such polypeptides and reference to “the agent” includes reference to one or more agents and equivalents thereof known to those skilled in the art, and so forth.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### EXAMPLES

The following Examples report the identification of polymorphisms in the human SORBS1 gene, analysis of variants, and the role of polymorphisms in hypertension and adipocyte differentiation. The Examples, which are intended to be purely exemplary of the invention and should therefore not be considered to limit the invention in any way, also describe and detail aspects and embodiments of the invention discussed above.

### Example 1

### Identification of Polymorphisms

SNPs were identified by screening all of the exons, and 50-150 base pairs of the flanking regions of the introns of the SORBS1 gene. Primers, based on the sequences at the intron/exon junctions, were used in polymerase chain reactions (PCR) to amplify patient genomic DNA. Forty chromosomes from 20 type 2 diabetic patients, 10 of whom were obese, and 10 of whom were not obese, were amplified and sequenced. Serum glucose levels were measured with an autoanalyzer (Hitachi 7250, Tokyo, Japan). Serum insulin levels were measured by a microparticle enzyme immunoassay using the AxSYM system from Abbott Diagnostics (Abbott Laboratories, Dainabot Co. Ltd., Japan). Type 2 diabetes was defined by the 1998 WHO criteria (Alberti, 1998). Obesity was defined as BMI greater than or equal to 30 kg/m² (Lin et al, 2001b). The primers used are listed in Table 1.

TABLE 1

Primers Used in DNA Sequencing Reactions to Screen the SORBS1 Gene

| | | Forward primer sequence | Reverse primer sequence |
|---|---|---|---|
| Exons | Size (bp) | (5′→3′) | (5′→3′) |
| 1 | 354 | GGTGACTCCTGGTTCAGCTCTG | ACCATGAAGGTGATGAGAGTGATG |
| 2 | 401 | CAGAGGGCCTCATAGGAACCAAG | CAACAACAGGCTCCAGGTCTCAG |
| 3 | 401 | GTGTGGGCGACTTCCTCCTATTG | AGATTCGCTGGCTCTCAGGAAAC |
| 4 | 404 | AGACTTCGCATGGCTGTAACCAG | GGGGACCGTAAGAGACACACAC |
| 5, 6 | 467 | GGGCGCTGGATTTTCAACCTTCTA | GTTATCCAACTGGGGAAGCAGGA |
| 7 | 518 | TACCTCACTGCATGCCCACTCTC | GACTGCTGGGAGGAGACATTCAGAA |
| 8 | 470 | TCCGCCCCATACTAATACCAACAA | CCTTGGGAAACAATGGGATTTGT |
| 9 | 383 | TAATGGGAGCCACGCTAATACCC | AGCTCCAGGGAGGCAGGAACTAT |
| 10 | 412 | GTTCAGGAGCGTTTTGTTTTGCAG | ACCCACTTTGGGAAAAGCTGTTGT |
| 11 | 416 | TGAGGTTTGGGACTCTTGGTTTTTC | CTTCCTCCCTCCTGTGCTCTGA |
| 12 | 399 | AGCCTGTTCATGGCAATTCTTTG | GCCCAAATCTTATGCCTACAATGG |
| 13 | 369 | CGAAAGAGTCAATTGGGATTTGTCA | CACTGGCTACCCCGTTAATCATGT |
| 14 | 353 | TCAGCAGCCCAGAGTGGTAATCC | CACAGAGTCAGGAAGGGGAAAGG |
| 15 | 396 | GCGTGGGTGAGATTTCTCTACTG | GGCGTCTGAAGAACTGAATGTGC |
| 16, 17 | 456 | ACCAGACTGTGGAAATGGGGAAC | TGGAGGCTCCTAGGTCTCAATGAA |
| 18 | 370 | CAAGCCTTGTGAGAAGCGAACAC | TTAAAGAACATGGGCACGGTGAG |
| 19 | 352 | ATTGCTGCTGTTGAATGCAGCTCC | GACAATCCCACTACGGACAACCA |
| 20 | 369 | TCCAACGATGATTTGCTCTTGAAAC | CTAGAGAGCACACACAGTCCAAC |
| 21 | 368 | TCATGCTATCTTGGCCGTATGAAAG | GTCAGAAAGCCATGCAGCCTAAAA |
| 22 | 288 | CTGACTTCCAGAGGTCCTGGTC | AACAAAACACACTCTGGGTCCAAAA |
| 23 | 383 | CCTCAGCAGGTAATGTGCTGTTTG | CAAGGCAGGACTTATCCCCATTG |
| 24 | 375 | GTGAACTCTTTTCGCAAGCTGGTT | AAGCTAGTGCCTGTGGGGAAATC |
| 25 | 410 | CTTAAAGCTTCTGCCCCATTCACC | AAGGGAGGCCAGGATGACAGATG |
| 26 | 389 | GTGAAAAGTGTCCTTGTTGTTCATG | CAACTCTATCACACCAGCTTGAGC |
| 27 | 418 | ATATGGAGGCCCCTCTCTGGAAT | TCAGCAGGAGGAAGAAGCTGAGA |
| 28 | 386 | AATCTTCCCACGCACCTACATCG | GTGTGATAGCAACACCACGCTTC |
| 29 | 389 | CATGCTAGGACAAGGACCCTCTC | ATTACAGGCATGAGCCACCACAC |
| 30 | 546 | CACCAGAGTGTCCTTGCTCATCC | ATCTTGGTGGATGCCTGAAGATG |
| 30 | 545 | ACATGAATGGAGACGGTGGTGTC | CTGAGGCAAGCAAATGGTCTCAC |
| 31 | 349 | TATCTGGTGTCATCCAAGCGTGA | GGGGTAATGCAGTGGCCTAACAT |
| 32 | 410 | CAGCACTGTTTCTCATCCCCTTG | CCAGTCAGCACAGCTCTCATTTG |
| 33 | 386 | GAATGCATCTCCCCAAATGCTAAG | GTGCAGAGGAGATTGCTTTGCTTT |
| 34 | 410 | ACAAGGGGTTTTGGAGCAGAAATC | GCTCCCAGAAGCGGCTAAGTAAA |

Primers were designed based on the intron/exon boundaries of the SORBS1 gene (Lin 2001b), and used to amplify genomic DNA using the polymerase chain reaction (PCR) method. Total genomic DNA was purified from peripheral blood leukocytes using the DNA extraction kit of Puregene® (Minneapolis, Minn.). Genomic DNA (20 ng), Tris-HCl pH 9.1 (50 mM), ammonium sulfate (16 mM), MgCl₂ (3.5 mM), bovine serum albumin (BSA) (4.5 μg), primer (6 pmol), dNTP (0.2 mM), and Klentaq® DNA polymerase (1.2 u) were combined in a total reaction volume of 30 μl. PCR was performed with initial denaturation at 96° C. for 2 min, and then with 35 cycles of 1 min. at 94° C., 55 sec. at 65° C., and 1 min. at 72° C., then a final 10 min. extension at 72° C.

The resulting PCR products were purified with a GFX PCR DNA purification kit (Amersham Pharmacia, Piscataway, N.J.) and then sequenced with PCR primers, as listed in Table 1, using Big Dye Terminator® chemistry, except for exons 15, 16, 25, and 32, which were sequenced with the primers exon 156P (5′-TTGGTTCT CTTACCTTAGCCAG-3′) exon 25P (5′-GGAGGCAAATAGGCAAATATTGC-3′), and exon 32P (5′-GCTGATGCAGMCATGGCCT-3′). Sequencing a total of 13,136 base pairs resulted in the identification of fourteen variants, which were identified with the programs in Biology Workbench 3.2 (http://workbench.sdsc.edu). They are listed in Table 2.

TABLE 2

SORBS1 Gene Polymorphisms

TABLE 2A: SORBS1 Gene Polymorphisms (Forward Primers)

| | | | | | |
|---|---|---|---|---|---|
| Location | Position | Forward Primer | SEQ ID NO | Common | Variant |

| | | | | | |
|---|---|---|---|---|---|
| exon 4 | 220 | AGACTTCGCATGGCTGTAACCAG | 1 | G | T |
| exon 4 | 249 | AGACTTCGCATGGCTGTAACCAG | 1 | A | G |
| exon 5 | −7 | GGGCGCTGGATTTTCAACCTTCTA | 3 | C | T |
| exon 6 | −25 | GGGCGCTGGATTTTCAACCTTCTA | 3 | A | G |
| exon 7 | 682 | GACTGCTGGGAGGAGACATTCAGAA | 5 | A | G |
| exon 9 | +64 | TAATGGGAGCCACGCTAATACCC | 7 | C | T |
| exon 10 | +61 | GTTCAGGAGCGTTTTGTTTTGCAG | 9 | C | T |
| exon 11 | +69 | TGAGGTTTGGGACTCTTGGTTTTTC | 11 | T | C |
| exon 16 | +33 | CAAGCCTTGTGAGAAGCGAACAC | 13 | C | T |
| exon 18 | 1482 | CAAGCCTTGTGAGAAGCGAACAC | 15 | T | C |
| exon 18 | 1518 | CAAGCCTTGTGAGAAGCGAACAC | 15 | C | T |
| exon 22 | −6 | CTGACTTCCAGAGGTCCTGGTC | 17 | G | T |
| exon 24 | +79 | GTGAACTCTTTTCGCAAGCTGGTT | 19 | C | T |
| exon 26 | 2337 | GTGAAAAGTGTCCTTGTTGTTCATG | 21 | G | A |

Table 2B: SORBS1 Gene Polymorphisms (Reverse Primers)

| | | | | | |
|---|---|---|---|---|---|
| Location | Position | Reverse Primer | SEQ ID NO | Common | Variant |

| | | | | | |
|---|---|---|---|---|---|
| exon 4 | 220 | GGGGACCGTAAGAGACACACAC | 2 | G | T |
| exon 4 | 249 | GGGGACCGTAAGAGACACACAC | 2 | A | G |
| exon 5 | −7 | GTTATCCAACTGGGGAAGCAGGA | 4 | C | T |
| exon 6 | −25 | GTTATCCAACTGGGGAAGCAGGA | 4 | A | G |
| exon 7 | 682 | GACTGCTGGGAGGAGACATTCAGAA | 6 | A | G |
| exon 9 | +64 | AGCTCCAGGGAGGCAGGAACTAT | 8 | C | T |
| exon 10 | +61 | ACCCACTTTGGGAAAAGCTGTTGT | 10 | C | T |
| exon 11 | +69 | CTTCCTCCCTCCTGTGCTCTGA | 12 | T | C |
| exon 16 | +33 | TGGAGGCTCCTAGGTCTCAATGAA | 14 | C | T |
| exon 18 | 1482 | TTAAAGAACATGGGCACGGTGAG | 16 | T | C |
| exon 18 | 1518 | TTAAAGAACATGGGCACGGTGAG | 16 | C | T |
| exon 22 | −6 | TTAAAGAACATGGGCACGGTGAG | 18 | G | T |
| exon 24 | +79 | AAGCTAGTGCCTGTGGGGAAATC | 20 | C | T |
| exon 26 | 2337 | CAACTCTATCACACCAGCTTGAGC | 22 | G | A |

A (+) or (−) in the position column indicates the position with respect to the intron/exon boundary.

Among the 14 SNPs identified, two affected amino acid coding, four occurred within exons without a change in amino acid coding, and the remaining eight occurred within introns. The two molecular variants that affected amino acid coding were R74W, present in exon 4, and consisted of a change from CGG to TGG, resulting in substitution of arginine with tryptophan; and T228A, present in exon 7, and consisted of a change from ACG to TCG, resulting in substitution of threonine with alanine.

### Example 2

### PCR-RFLP Analysis of T228A Variants

Polymorphism T228A of nucleotide 682 in exon 7 of SORBS1 showed a significant association with human obesity and type 2 diabetes (Lin 2001b). The T228 variants were analyzed using restriction fragment length polymorphism analysis combined with PCR. Genomic DNA (10 ng), Tris-HCl pH 8.3 (10 mM), KCl (50 mM), MgCl₂ (15 mM), exon 7 forward primer (5′-GACTGCTGGGAGGAGACATTCAGAA-3′) (SEQ ID #5) (0.2 μM), exon 7 reverse primer (5′-GACTGCTGGGAGGAGACATTCA GM-3′) (SEQ ID #6) (0.2 μM), dNTP (0.2 mM), and KlenTaq polymerase (0.6 u) were combined in a total reaction volume of 15 μl. PCR was performed with initial denaturation at 96° C. for 2 min, and then with 35 cycles of 30 sec. at 94° C., 30 sec. at 65° C., and 30 sec. at 72° C., then a final 10 min. extension at 72° C.

The resulting PCR product was digested with KasI restriction enzyme for genotyping. Digested DNA fragments were separated on a 1.5% agarose gel. A single uncut fragment (518 bp) indicated a genotype of T/T. The presence of two fragments of 360 and 158 bp indicated a genotype of A/A. The presence of three fragments of 518, 360, and 158 hp indicated a genotype of T/A.

Testing these specific variants in the SORBS1 gene demonstrated that they are involved with the regulation of insulin metabolism. The T228A variant affected insulin resistance by exerting a primary effect on the body mass index (BMI), a measure of obesity. Individuals with the TT phenotype had a higher BMI than individuals with the TA or AA phenotype (30.1±0.6 versus 27.6±1.0 kg/m², p=0.041). Individuals with the TT phenotype also had a higher fasting plasma insulin concentration than individuals with the TA or AA phenotype (11±1 versus 8±1 mU/l, p=0.025). Individuals with the TT phenotype also had a higher two hour plasma insulin concentration after oral glucose challenge than individuals with the TA or AA phenotype (51.5±5 versus 32±6 mU/l, p=0.039). Finally, individuals with the TT phenotype had a higher insulin resistance index, indicated by HOMA-IR, than individuals with the TA or AA phenotype (2.77±1.8 versus 1.96±0.24, p=0.022). No differences were observed in fasting plasma glucose concentration, or two hour plasma glucose concentration after oral glucose challenge.

The A allele of the T228A polymorphism is a protective marker for both obesity and diabetes. The frequency of heterozygous or homozygous variants (T/A or A/A genotype) were significantly lower in obese (15%, p=0.02) and diabetic (19.2%, p=0.05) populations as compared to normal controls (27.2%). The non-obese study group had almost twice the frequency of the variant A allele as the obese group (14.9% versus 7.5%, p=0.007).

The genotypic frequency and allele frequency of the T228A polymorphism was also studied in a cohort of 1796 patients from 740 families of Chinese and Japanese origins, recruited in the San Francisco Bay Area, Hawaii, and Taiwan. This cohort included hypertensive probands, their parents, and their siblings.

The genotype and allele frequencies of T228A polymorphism of the SORBS1 gene in Japanese and Chinese patients were significantly different (p<0.0001). Among all Chinese study participants, including hypertensive and non-hypertensive, 75.5% had the TT genotype, 23.3% had the TA genotype, and 1.20% had the AA genotype. Among all Japanese study participants, 88.98% had the TT genotype, 10.15% had the TA genotype, and 0.86% had the AA genotype. The allelic frequency of the A allele was 12.85% in the Chinese patients, and 5.94% in the Japanese patients.

The genotype and allele frequencies of T228A polymorphism in the hypertensive probands between Japanese and Chinese patients were also significantly different (p<0.0001). Among Chinese hypertensive probands, 72.24% had the TT genotype, 27.03% had the TA genotype, and 0.74% had the AA genotype. Among all Japanese hypertensive probands, 92.62% had the TT genotype, 6.71% had the TA genotype, and 0.67% had the AA genotype. The allelic frequency of the A allele was 14.25% in the Chinese hypertensive probands, and 4.03% in the Japanese hypertensive probands.

### Example 3

### Role of T228A in Blood Pressure Regulation

Polymorphism T228A of nucleotide 682 in exon 7 of SORBS1 showed a significant association with blood pressure regulation and hypertension. Individuals were recruited as part of the Stanford, Asia, Pacific Program for Hypertension and Insulin Resistance (SAPPHIRe) to undergo genotype analysis with respect to the T228A of the SORBS1 gene. Details of this study population have been published elsewhere (Ranade, et al., 2000a). A total of 1713 patients from 602 families of Japanese or Chinese descent, including hypertensive probands, their parents, and their siblings, underwent genotype analysis. Resting systolic and diastolic blood pressure (SBP and DBP), mean arterial pressure (MAP) and pulse rate (PR) were determined by use of an oscillometric device, the Dinamap model 1846 SX (Critikon). Total genomic DNA was purified from peripheral blood leukocytes using the Puregene DNA extraction kit (Minneapolis, Minn.). The same primers and conditions for PCR-RFLP genotype analysis of T228A of the SORBS1 gene were employed as reported in Example 2.

Comparing the phenotypic variables and genotypes of siblings from 11 families demonstrated that subjects with the genotype homozygous AA had significantly lower diastolic blood pressure and mean arterial pressure than that of their siblings with genotype TT or TA after adjustment for age, gender and BMI. The difference in systolic blood pressure was statistically borderline. AA homozygotes had a diastolic blood pressure 7 mmHg lower, and a mean arterial pressure 10 mmHg lower than their siblings with a TT or TA genotype. A borderline lower level of systolic blood pressure (10 mmHg) was also observed for subjects with AA vs. TT and TA genotypes.

TABLE 3
COmparisons Between Subjects With AA Vs. TT Or TA Genotypes
After Adjustment For Age, Gender And BMI

| | | | TT + TA vs. AA |
|---|---|---|---|
| | TT + TA | AA | 602 families |
| N | 1697 | 19 | 1697 19 |
| Systolic BP | 132.22 ± 0.70 | 121.77 ± 6.01 | 0.0830 |
| Diastolic BP | 78.23 ± 0.38 | 71.03 ± 3.24 | 0.0269* |
| Pulse Rate | 68.56 ± 0.33 | 69.11 ± 2.77 | 0.8428 |
| Mean | | | |
| Arterial | 100.07 ± 0.50 | 89.62 ± 4.31 | 0.0158* |
| Pressure | | | |

As predicted by the observation that the T228A polymorphism showed a significant influence on blood pressure regulation, the risk for hypertension was significantly lower for siblings with the AA genotype after adjustment for age, gender and BMI (OR=0.18; p=0.0136). Multiple regression analysis to evaluate the relative contribution of SORBS1 genotype on blood pressure regulation and development of hypertension demonstrated a significant effect of the SORBS1 genotype on mean arterial pressure, diastolic blood pressure, and hypertension status. Such well-known factors associated with hypertension, as age, gender, BMI, dyslipidemia, and insulin resistance also demonstrated effects. A borderline effect of the SORBS1 genotype on systolic blood pressure regulation was also observed (p=0.07).

A sibling-based transmission-disequilibrium test further confirmed the contribution of the T228A polymorphism of the SORBS1 gene in hypertension. There was a significant linkage between the T228A polymorphism and hypertension (Z score=3.178, p=0.000742).

Most of the hypertensive patients in the study were under treatment with antihypertensive agents. An analysis of interactions between genotype with the medication categories demonstrated that among patients placed on beta blocking drugs, those with the TT genotype had higher diastolic blood pressures than those with TA or AA genotypes, indicating that beta blockers provide a more effective treatment option for patients with TA or AA genotypes compared to those with the TT genotype.

### Example 4

### Gene Expression in Adipocyte Differentiation

3T3-L1 cells (CL173, American Type Culture Collection) maintained in Dulbecco's modified Eagle's medium (DMEM) containing 10% calf serum can be induced to differentiate to adipocytes in culture, as previously described (MacDougald et al., 1994). Differentiation was induced by incubating confluent cells in DMEM medium containing 10% fetal bovine serum (FBS), 1 μM dexamethasone, 0.5 mM 3-isobutyl-1-methylxanthine and 10 μM insulin (MacDougald et al., 1994).

Messenger RNA differential display was performed with Gene Hunter® according to the manufacturer's instruction (Gene Hunter®, Nashville, Term.). Total RNA was isolated from undifferentiated cells (day −2), cells at the beginning of differentiation induction (day 1), and after completion of differentiation (day 10) using a phenol/guanidine thiocyanate-based protocol (TRIzol®, BRL, Gaithersburg, Md.). Total RNA (50 μg) from each RNA sample was then treated with 20 units of RNase-free DNase I, and 0.2 μg of the treated RNA were reverse transcribed, then amplified by PCR. The PCR reaction (20 μl total volume) was performed in 10 mM Tris-HCl, pH 8.3, 50 mM KCl, 1.5 mM MgCl₂, 0.01% gelatin, 2 μM dNTP (in the absence of dATP), 0.25 μl of γ-[³³P]dATP (2000 Ci/mmole), 0.2 μM 5′ arbitrary primer and 3′ anchored primer (dT), and 1 unit Vent® DNA polymerase (New England Biolabs, Beverly, Mass.). The reaction underwent 40 cycles of denaturing at 94° C. for 30 seconds, annealing at 40° C. for 2 min, and extension at 72° C. for 1 min; a final extension step at 72° C. for 5 min was added at the end of reaction. The PCR reaction mixture was resolved on a 6% LongRanger® denaturing polyacrylamide gel containing 7M urea, and differentially amplified PCR fragments were visualized by exposing the dried gel to x-ray film overnight at room temperature. The differentially expressed fragments were excised from the gel, and the DNA was recovered from the gel slices by boiling in 60 μl of water for 10 min. The eluted products were then re-amplified with the Clontech Advantage® PCR system. (Clontech, Palo Alto, Calif.). The re-amplified fragments were subsequently cloned into the TA cloning pGEM-Teasy® vector (Promega, Madison, Wis.) and sequenced with T7 and SP6 primers by an ABI PRISM® dye terminator cycle ready reaction kit (Perkin-Elmer, Foster City, Calif.). The DNA sequences obtained were entered into a personal computer and homology search was carried out with the NCBI BLAST-N program.

Using mRNA differential display analysis, and confirmed by Northern blot, 56 genes were identified as differentially expressed during differentiation of 3T3-L1 cells to adipocytes. Among these 56 genes, 40 were up-regulated and 16 were down-regulated during adipocyte differentiation.

Expression of the SH3P12 gene increased during differentiation. The level of expression was approximately twice as high at day 10 as on day 1 of induction. The increased level of the SH3P12 gene expression correlated with, and was immediately preceded by, an increase in expression of the transcriptional factor PPARγ.

The anti-diabetic agent BRL49653 (4.5 μM) was added to the culture medium, after the cells were differentiated, for 16 hours to study its effect on gene expression. In the absence of BRL49653, the level of SH3P12/CAP/ponsin mRNA on day 6 post-induction was 8.25 fold higher than preinduction levels. When BRL49653 was added to the well-differentiated adipocytes on day 6, the level of SH3P12/CAP/ponsin mRNA was further increased to 17.6 fold higher than pre-induction levels.

It will be apparent to those skilled in the art that various modifications and variations can be made in the practice of the present invention without departing from the scope or spirit of the invention. Other embodiments of the invention will be apparent to those skilled in the art from consideration of the specification and practice of the invention disclosed herein. It is intended that the specification and Examples be considered as exemplary only, with a true scope and spirit of the invention being indicated by the following claims.

### REFERENCES

The specification is most thoroughly understood in light of the following references, all of which are hereby incorporated in their entireties.

Alberti, K. G., Zimmet, P. Z. (1998) Definition, diagnosis and classification of diabetes mellitus and its complications. Part 1: Diagnosis and classification of diabetes mellitus provisional report of a WHO consultation. Diabet. Med. 15: 539-553.

Alberts, B, Bray, D., Lewis, J, Raff, M., Roberts, K., Watson, J. D., editors (1994) MOLECULAR BIOLOGY OF THE CELL, THIRD EDITION, 761-762.

Baumann, C. A., Ribon, V., Kanzaki, M., Thurmond, D. C., Mora, S., Shigematsu, S., Bickel, P. E., Pessin, J. E., Saltiel, A. R. (2000) CAP defines a second signaling pathway required for insulin-stimulated glucose transport. Nature 407:202-207.

Bickel, P. E., Scherer, P. E., Schnitzer, J. E., Oh, P., Lisanti, M. P., Lodish, H. F. (1997) Flotillin and epidermal surface antigen define a new family of caveolae-associated integral membrane proteins. J. Biol. Chem. 272:13793-13802.

Bjorntorp, P., Holm, G., Rosmond, R., Folkow, B. (2000) Hypertension and the metabolic syndrome: closely related central origin? Blood Press. 9:71-82.

Charpin, G., Chikh-Issa, A. R., Guignard, H., Jourdan, G., Dumas, C., Pansu, D., Descroix-Vagne, M. (1992) Effect of sorbin on duodenal absorption of water and electrolytes in the rat. Gastroenterology 103:1568-1573.

Collins, F. S., Guyer, M. S., Chakravarti, A. (1997) Variations on a theme: Cataloging human DNA sequence variation. Science 278:1580.

Hauner, H. (2002) Insulin resistance and the metabolic syndrome-A challenge of the new millennium. Eur. J. Clin. Nutr. 56 Suppl.:S25-29.

Hess, P., Cooper, D. (1999) Impact of pharmacogenetics on the clinical laboratory. Mol. Diagn. 4:289.

Kioka, N., Ueda, K., Amachi, T. (2002) Vinexin, CAP/ponsin, ArgBP2: A novel adaptor protein family regulating cytoskeletal organization and signal transduction. Cell Struct. Funct. 27:1-7.

Lin, W. H., Huang, C. J., Liu, M. W., Chang, H. M., Chen, Y. J., Tai, T. Y., Chuang, L. M. (2001a) Cloning, mapping, and characterization of the human sorbin and SH3 domain containing 1 (SORBS1) gene: A protein associated with c-Abl during insulin signaling in the hepatoma cell line Hep3B. Genomics 74:12-20.

Lin, W. S., Chang, H. M., Tai, T. Y., Chuang, L. M. (1999) Effect of thiazolidinedione in gene expression in NIH3T3-L1 adipocytes. Diabetes 48 (Suppl. 1): A217.

Lin, W. H., Chiu, K. C., Chang, H. M, Lee, K. C., Tai, T. Y., and Chuang, L. M. (2001b) Molecular scanning of the human sorbin and SH3-domain-containing-1 (SORBS1) gene: Positive association of the T228A polymorphism with obesity and type 2 diabetes. Human Molecular Genetics 17:1753-1760.

Lopez-Candales, A. (2001) Metabolic syndrome X: A comprehensive review of the pathophysiology and recommended therapy. J. Med. 32:283-300.

Kimura, A., Baumann, C. A., Chiang, S. H., Saltiel, A. R. (2001) The sorbin homology domain: a motif for the targeting of proteins to lipid rafts. Proc. Natl. Acad. Sci. 98:9098-9103.

MacDougald, O. A., Cornelius, P., Lin, F. T., Chen, S. S., Lane, M. D. (1994) Glucocorticoids reciprocally regulate expression of the CCMT/enhancer-binding protein alpha and delta genes in 3T3-L1 adipocytes and white adipose tissue. J. Biol. Chem. 269:19041-19047.

Mandai, K., Nakanishi, H., Satoh, A. Takahashi, K., Satoh, K., Nishioka, H., Mizoguchi, A., and Takai, Y. (1999) Ponsin/SH3P12: An I-afadin- and vinculin-binding protein localized at cell-cell and cell-matrix adherens junctions. J. Cell. Biol. 144:1001-1018.

Mayer, B. J. (2001) SH3 domains: Complexity in moderation. J. Cell. Sci. 114:1253-1263.

Nieters, A., Becker, N., Linseisen, J. (2002) Polymorphisms in candidate obesity genes and their interaction with dietary intake of n-6 polyunsaturated fatty acids affect obesity risk in a sub-sample of the EPIC-Heidelberg cohort. Eur. J. Nutr. 41:210-221.

Province, M. A., Boerwinkle, E., Chakravarti, A., Cooper, R., Formage, M., Leppert, M., Risch, N., Ranade, K. (2000) Lack of association of the angiotensinogen-6 polymorphism with blood pressure levels in the comprehensive NHLBI Family Blood Pressure Program. J. Hypertens. 18:867-876.

Ranade, K., Hsuing, A. C., Wu, K. D., Chang, M. S., Chen, Y. T., Hebert, J., Chen, Y. I., Olshen, R., Curb, D., Dzau, V., Botstein, D., Cox, D., Risch, N. (2000) Lack of evidence for an association between alpha-adducin and blood pressure regulation in Asian populations. Am. J. Hypertens. 13:704-709.

Ranade, K., Shue, W. H., Hung, Y. J., Hsuing, C. A., Chiang, F. T., Pesich, R., Hebert, J., Olivier, M., Chen, Y. D., Pratt, R., Olshen, R., Curb, D., Botstein, D., Risch, N., Cox, D. R. (2001a) The glycine allele of a glycine/arginine polymorphism in the beta2-adrenergic receptor gene is associated with essential hypertension in a population of Chinese origin. Am. J. Hypertens. 14:1196-1200.

Ranade, K., Wu, K. D., Hwu, C. M., Ting, C. T., Pei, D., Pesich, R., Hebert, J., Chen, Y. D., Pratt, R., Olshen, R., Masaki, K., Risch, N., Cox, D. R., Botstein, D. (2001b) Genetic variation in the human urea transporter-2 is associated with variation in blood pressure. Hum. Mol. Genet. 10:2157-2164.

Ribon, V., Johnson, J. H., Camp, H. S., Saltiel, A. R. (1998b) Thiazolidinediones and insulin resistance: Peroxisome proliferator-activated receptor γ activation stimulates expression of the CAP gene. Proc. Natl. Acad. Sci. 95:14751-14756.

Ribon, V., Printen, J. A., Hoffman, N. G., Kay, B. K., Saltiel, A. R. (1998a) A novel, multifunctional c-Cbl binding protein in insulin receptor signaling in 3T3-L1 adipocytes. Mol. Cell. Biol. 18:872-879.

Simons, K., Toomre, D. (2000) Lipid rafts and signal transduction. Nat. Rev. Mol. Cell. Biol. 1:31-39.

Sparks, A. B., Hoffman, N. G., McConnell, S. J., Fowlkes, D. M., Kay, B. K. (1996) Cloning of ligand targets: systematic isolation of SH3 domain-containing proteins. Nat. Biotechnol. 14:741-744.

Wiltshire, S., Hattersley, A. T., Hitman, G. A., Walker, M., Levy, J. C., Sampson, M., O'Rahilly, S., Frayling, T. M., Bell, J. I., Lathrop, G. M., Bennett, A., Dhillon, R., Fletcher, C., Groves, C. J., Jones, E., Prestwich, P., Simecek, N., Rao, P. V. S., Wishart, M., Foxon, R., Howell, S., Smedley, D., Cardon, L. R., Menzel, S., McCarthy, M. I. (2001) A Genomewide Scan for Loci Predisposing to Type 2 Diabetes in a U.K. Population (The Diabetes UK Warren 2 Repository): Analysis of 573 Pedigrees Provides Independent Replication of a Susceptibility Locus on Chromosome 1q. Am. J. Hum. Genet. 69:553-569.

Yang, W. S., Lee, W. J., Huang, K. C., Lee, K. C., Chao, C. L., Chen, C. L., Tai, T. Y., Chuang, L. M. (2003) mRNA Levels of the Insulin-Signaling Molecule SORBS1 in the Adipose Depots of Nondiabetic Women. Obes. Res. 1:586-590.

Zhang, M., Kimura, A., Saltiel, A. R. (2003) Cloning and characterization of Cbl-associated protein splicing isoforms. Mol. Med. 9:18-25.

What is claimed is:

1. A method of detecting at least one single nucleotide polymorphism in a human sorbin and SH3-domain-containing-1 (SORBS1) gene, which comprises determining the nucleotide present at one or more positions chosen from 220; 249; −7 with respect to exon 5; −25 with respect to exon 6; 682; +64 with respect to exon 9; +61 with respect to exon 10; +69 with respect to exon 11; +33 with respect to exon 16; 1482; 1518; −6 with respect to exon 22; +79 with respect to exon 24; and 2337.

2. The method of claim 1, wherein the SNP at position 220 is the presence of G and/or T.

3. The method of claim 1, wherein the SNP at position 249 is the presence of A and/or G.

4. The method of claim 1, wherein the SNP at position −7 with respect to exon 5 is the presence of C and/or T.

5. The method of claim 1, wherein the SNP at position −25 with respect to exon 6 is the presence of A and/or G.

6. The method of claim 1, wherein the SNP at position 682 is the presence of A and/or G.

7. The method of claim 1, wherein the SNP at position +64 with respect to exon 9 is the presence of C and/or T.

8. The method of claim 1, wherein the SNP at position +61 with respect to exon 10 is the presence of C and/or T.

9. The method of claim 1, wherein the SNP at position +69 with respect to exon 11 is the presence of T and/or C.

10. The method of claim 1, wherein the SNP at position +33 with respect to exon 16 is the presence of C and/or T.

11. The method of claim 1, wherein the SNP at position 1482 is the presence of T and/or C.

12. The method of claim 1, wherein the SNP at position 1518 is the presence of C and/or T.

13. The method of claim 1, wherein the SNP at position −6 with respect to exon 22 is the presence of G and/or T.

14. The method of claim 1, wherein the SNP at position +79 with respect to exon 24 is the presence of C and/or T.

15. The method of claim 1, wherein the SNP at position 2337 is the presence of G and/or A.

16. The method of any of claims 1-15 wherein the sequence is determined by amplification and sequencing of the SORBS1 gene using one or more of the the primers defined by SEQ ID NO 1-SEQ ID NO 22.

17. A method of associating one or more SORBS1 SNPs with an insulin disorder, comprising determining the nucleotide present at one or more of positions 220; 249; −7 with respect to exon 5; −25 with respect to exon 6; 682; +64 with respect to exon 9; +61 with respect to exon 10; +69 with respect to exon 11; +33 with respect to exon 16; 1482; 1518; −6 with respect to exon 22; +79 with respect to exon 24; and 2337 in the SORBS1 gene; and correlating the nucleotide with the presence or absence of the insulin disorder.

18. The method of claim 17, wherein the partial or complete sequence of the SORBS1 gene is determined by amplification and sequencing of the SORBS1 gene using the primers defined by SEQ ID NO 1-SEQ ID NO 22.

19. The method of claim 17, wherein the insulin disorder is selected from type 2 diabetes, obesity, hypertension, atherosclerosis, and metabolic syndrome.

20. A method for determining whether an individual is at increased or decreased risk for an insulin disorder, comprising obtaining a sample from a patient, and determining the presence or absence in the sample of one or more SORBS1 SNPs.

21. The method of claim 20, wherein the SNP encodes a T228A polymorphism.

22. A kit for the identification of mutations in the SORBS1 gene in a patient sample, comprising at least one primer pair for amplification of an exon of the SORBS1 gene, each member of said primer pair being labeled with a detectable label, wherein the kit comprises:
(a) a primer pair selected from SEQ ID NO 1-SEQ ID NO 22; and
(b) instructions for performing an assay to detect the human SORBS1 gene in the sample.
